(19) United States
(12) Plant Patent Application Publication
(10) Pub. No.: US 2015/0334894 P1
(43) Pub. Date: Nov. 19, 2015

(54) Dianthus plant named 'KLEDP13122'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/120,256

(22) Filed: May 13, 2014

(51) Int. Cl. A01H 5/00

(52) U.S. Cl. PLT/272

(57) ABSTRACT

A new Dianthus plant particularly distinguished by double, violet bi-color flowers and an upright form, is disclosed.

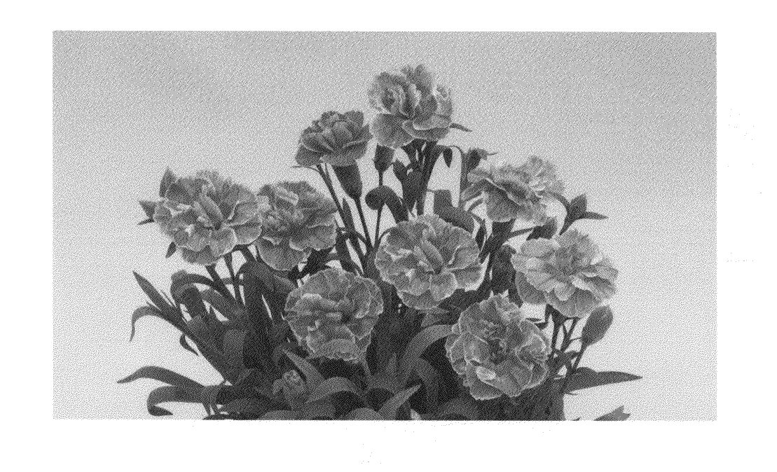

### GENUS AND SPECIES

Dianthus caryophyllus

### VARIETY DENOMINATION

‘KLEDP13122’

### BACKGROUND OF THE NEW PLANT

The present invention comprises a new and distinct variety of Dianthus, botanically known as Dianthus caryophyllus, and hereinafter referred to by the variety name ‘KLEDP13122’. ‘KLEDP13122’ originated from a cross-pollination conducted in June 2006 in Stuttgart, Germany between the proprietary female Dianthus variety ‘DCP 2004 0017’ (unpatented) and the proprietary male Dianthus variety ‘DCP 2005 0139’ (unpatented).

The seeds from the cross were sown and plants were grown in a greenhouse for evaluation, where an individual plant designated ‘KLEDP13122’ was selected from the group of plants in May 2006 in Stuttgart, Germany. In June 2007, ‘KLEDP13122’ was first vegetatively propagated by vegetative cuttings. ‘KLEDP13122’ was found to reproduce true to type in successive generations of asexual propagation in Stuttgart, Germany via vegetative cuttings.

### SUMMARY

The following are the most outstanding and distinguishing characteristics of this new variety when grown under normal horticultural practices in Stuttgart, Germany.

1. Double, violet bi-color flowers; and

2. An upright form.

### CROSS REFERENCE TO RELATED APPLICATIONS

Plant Breeders Rights for this variety have been filed with the European Community Plant Variety Rights Office on Jun. 28, 2013, Application No. 2013/1746. ‘KLEDP13122’ has not been sold or made publicly available more than one year prior to the filing of the instant application.

### DESCRIPTION OF THE PHOTOGRAPH

This new Dianthus plant is illustrated by the accompanying photograph of a plant approximately 5-month old and grown in a glass greenhouse in Stuttgart, Germany. The photograph shows the whole plant, including the flowers and foliage and buds and was taken in April 2011 in Stuttgart, Germany. The colors shown are as true as can be reasonably obtained by conventional photographic procedures.

### DESCRIPTION OF THE NEW VARIETY

The following detailed descriptions set forth the distinctive characteristics of ‘KLEDP13122’. The data which define these characteristics were collected from asexual reproductions carried out in Stuttgart, Germany. Data was collected on 5-month old plants grown in a greenhouse. Color references are to The R.H.S. Colour Chart of The Royal Horticultural Society of London (R.H.S.), 5^{th} edition (2007).
Classification:
   Family.—Caryophyllaceae.
   Botanical.—Dianthus caryophyllus.
   Common.—Pot carnation.
   Designation.—‘KLEDP13122’.
Parentage:
   Female parent.—The proprietary female Dianthus variety ‘DCP 2004 0017’ (unpatented).
   Male parent.—The proprietary male Dianthus variety ‘DCP 2005 0139’ (unpatented).
Plant:
   Propagation type.—Vegetative cuttings.
   Time to produce a rooted young plant.—14 to 16 days.
   Root description.—White, fine.
   Rooting habit.—Lanceolate.
   Plant form.—Potted plant, upright.
   Plant height.—13.0 cm to 15.0 cm.
   Plant diameter or spread.—16.0 cm to 18.0 cm.
Lateral branches:
   Length.—8.0 cm to 10.0 cm.
   Diameter.—4.0 mm to 6.0 mm.
   Internode length.—0.7 cm to 0.9 cm.
   Node length.—0.4 cm to 0.5 cm.
   Node diameter.—0.5 cm.
   Texture.—Smooth.
   Color.—RHS 137A.
Foliage:
   Arrangement.—Opposite.
   Length.—4.0 cm to 6.0 cm.
   Width.—7.0 cm to 9.0 cm.
   Shape.—Lanceolate.
   Apex.—Acute.
   Base.—Obtuse.
   Margin.—Entire.
   Texture (both upper and lower surfaces).—Smooth.
   Immature leaf color.—Upper surface: RHS 137C Lower surface: RHS 138A.
   Mature leaf color.—Upper surface: RHS 139A Lower surface: RHS 137A.
   Venation pattern.—Palmate.
   Venation color (both upper and lower surfaces).—RHS 139A.
Flowers:
   Flower type and habit.—Double, round, and small sprays.
   Fragrance.—Present, strong.
   Natural flowering season.—Spring to fall.
   Flower longevity on the plant.—10 to 12 days.
   Buds.—Length: 1.6 cm to 1.8 cm Diameter: 0.8 cm to 1.0 cm Shape: Elliptic Color: RHS 143C and RHS 183A.
   Corolla.—Quantity of petals and arrangement: 10 to 12 petals arranged in several whorls Corolla height: 1.5 cm to 2.0 cm Petal length: 3.5 cm Petal width: 2.0 cm to 2.5 cm Petal shape: Spoon-shaped Petal apex: Obtuse Petal base: Acute Petal margin: Crenate Petal texture (both upper and lower surfaces): Smooth Petal color: Immature: Upper surface: RHS N78A and RHS N58B Lower surface: RHS N78C Mature: Upper surface: RHS 76B and RHS 72B Lower surface: RHS 69C.
   Calyx.—Quantity of sepals and arrangement: Composed of 5 fused sepals Calyx height: 1.6 cm to 1.8 cm Calyx diameter (at the apex): 0.8 cm to 1.8 cm Sepal texture (both upper and lower surfaces): Smooth Sepal color: Upper surface: RHS N137C Lower surface: RHS 144A.
Peduncles:
   Length.—12.0 cm to 14.0 cm.
   Diameter.—0.2 cm.
   Strength.—Moderate.
   Texture.—Smooth.
   Color.—RHS N137B.
Reproductive organs:
   Stamens.—Quantity per flower: 9 to 11 Filament length: 2.5 cm Filament color: RHS 73A Anther length: 0.2 cm Anther diameter: 0.1 cm Anther color: RHS 93D Pollen amount: Abundant.
   Pistils.—Quantity per flower: 1 Pistil length: 0.1 cm Style length: 1.8 cm Style color: RHS 49D Stigma shape: Filamentous Stigma color: RHS 73B Ovary size: 0.5 cm Ovary shape: Oval Ovary color: RHS 145B.
Fruit and seed set: No fruit and seed set observed.
Disease and pest/insect resistance: No disease and pest/insect resistance observed.

### COMPARISON WITH PARENTAL LINES AND KNOWN VARIETY

‘KLEDP13122’ is a distinct variety of Dianthus and is distinguished from its parents as described in Table 1.

TABLE 1
Comparison with Parental Lines

| | | Female Parent | Male Parent |
|---|---|---|---|
| Characteristic | ‘KLEDP13122’ | ‘DCP 2004 0017’ | ‘DCP 2005 0139’ |
| Flower color | Violet bicolor | Purple | Lavender |

‘KLEDP13122’ is a distinct variety of Dianthus and is most similar to the commercial Dianthus variety SuperTrouper™ ‘Carmen’ (unpatented). Differences between the varieties are described in Table 2.

TABLE 2
Comparison with Similar Variety

| Characteristic | ‘KLEDP13122’ | SuperTrouper ™ ‘Carmen’ |
|---|---|---|
| Flower type | Double | Single |
| Flower color | Violet bicolor | Dark-red bicolor |

We claim:

1. A new and distinct variety of Dianthus plant designated ‘KLEDP13122’ as shown and described herein.
